(19) United States
(12) Patent Application Publication
(10) Pub. No.: US 2021/0404210 A1
(43) Pub. Date: Dec. 30, 2021

(54) GERMICIDAL DOOR HANDLE

(71) Applicant: ASSA ABLOY Access and Egress Hardware Group, Inc., New Haven, CT (US)

(72) Inventor: Daniel W. Riley, Meriden, CT (US)

(73) Assignee: ASSA ABLOY Access and Egress Hardware Group, Inc., New Haven, CT (US)

(21) Appl. No.: 17/352,426

(22) Filed: Jun. 21, 2021

Related U.S. Application Data
(60) Provisional application No. 63/046,576, filed on Jun. 30, 2020.

(51) Int. Cl. E05B 1/00; A61L 2/08; E05B 17/10

(52) U.S. Cl. CPC E05B 1/0069; E05B 17/10; A61L 2/084

(57) ABSTRACT

A germicidal door handle or other surface touched by a user may include one or more light sources configured to emit light at a wavelength effective to act as a germicidal. In some instances, the door handle/surface may interact with a controller configured to selectively activate the one or more light sources. The controller may be configured to perform the following steps: (1) activating the one or more light sources, (2) deactivating the one or more light sources upon an occurrence of a first predetermined condition, and (3) reactivating the one or more light sources upon an occurrence of a second predetermined condition.

### CROSS-REFERENCE TO RELATED APPLICATIONS

This Application claims the benefit under 35 U.S.C. § 119(e) of U.S. Provisional Application Ser. No. 63/046,576, titled “GERMICIDAL DOOR HANDLE,” filed on Jun. 30, 2020, which is incorporated herein in its entirety.

### FIELD

Disclosed embodiments relate to door actuators configured to include a germicidal, for example to inhibit the growth or action of microorganisms.

### BACKGROUND

Door handles (e.g., a door knob, a lever, a push bar, or other suitable door handle) may be used to open a door. Generally speaking, door handles may be used in public settings to allow, for example, individuals to enter a public building. However, as multiple people use a door handle in a public setting, the door handle may retain microorganisms, some of which may be disease causing. As multiple people use a given door handle, those people may deposit and/or come into contact with the microorganisms retained on the door handle. Thus, a door handle that retains microorganisms, particularly a door handle used in a public setting, may facilitate the transfer of diseases such as COVID-19.

### BRIEF SUMMARY

According to one embodiment, a door handle lever includes a body, one or more light sources mounted in the body, and a transparent grip sleeve surrounding the one or more light sources. The one or more light sources may be configured to emit light at a wavelength effective to act as a germicidal.

According to another embodiment, a door exit device includes a door handle, a locking mechanism, a body, one or more light sources mounted in the body, a transparent grip sleeve surrounding the one or more light sources, and a controller. The controller may be disposed on the locking mechanism and may be configured to perform the following steps: (1) activating the one or more light sources, (2) deactivating the one or more light sources upon an occurrence of a first predetermined condition, and (3) reactivating the one or more light sources upon an occurrence of a second predetermined condition. The one or more light sources may be configured to emit light at a wavelength effective to act as a germicidal when active.

According to another embodiment, an upgrade kit for a door handle lever includes a body, one or more light sources configured to be mounted in the body, a transparent grip sleeve configured to surround the one or more light sources, a portable power supply configured to provide electrical power to the one or more light sources, and a controller disposed on the body. The controller may be configured to perform the following steps: (1) activating the one or more light sources, (2) deactivating the one or more light sources upon an occurrence of a first predetermined condition, and (3) reactivating the one or more light sources upon an occurrence of a second predetermined condition. The one or more light sources may be configured to emit light at a wavelength effective to act as a germicidal when active.

According to another embodiment, An access control system for an entryway, the system comprising: a surface configured to be touched by a user and configured to assist in access through the entryway; one or more light sources mounted adjacent to the surface; wherein the one or more light sources are configured to emit light onto the surface at a wavelength effective to act as a germicidal.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

FIG. 1 is a perspective view of a germicidal door handle lever according to one illustrative embodiment;

FIG. 2 is a perspective view of a germicidal door handle lever according to one illustrative embodiment;

FIG. 3 is a front view of a door with a germicidal door exit device according to one illustrative embodiment;

FIG. 4 is a perspective view of the functional components of a germicidal door exit device according to one illustrative embodiment;

FIG. 5 is a perspective view of the functional components of a germicidal door exit device according to one illustrative embodiment;

FIG. 6 is a perspective view of an upgrade kit for a germicidal door handle lever according to one illustrative embodiment;

FIG. 7 is a perspective view of an upgrade kit for a germicidal door handle lever according to one illustrative embodiment;

FIG. 8A is a graph showing the path of light emitted by a germicidal door handle lever according to one illustrative embodiment;

FIG. 8B is a graph showing a plurality of paths of light emitted by a germicidal door handle lever according to one illustrative embodiment;

FIG. 9 is a flowchart showing the logic of a controller for a germicidal door handle according to one illustrative embodiment; and

FIG. 10 is a side view of a germicidal door handle lever according to one illustrative embodiment.

### DETAILED DESCRIPTION

Door handles (e.g., a door knob, a lever, a push bar, or other suitable door handle) may be mounted on doors used in public settings to allow individuals to access through doorways to publicly accessible buildings (e.g., a store front, an office building, a restaurant or any other suitable publicly accessible building). However, as multiple individuals touch a door handle in a public setting, those individuals may transfer microorganisms onto the door handle and/or come into contact with microorganisms already retained on the door handle. Some microorganisms may serve to cause diseases, such as COVID-19, the flu, or other illnesses. Thus, as a given door handle is used by multiple individuals, the risk of a subsequent individual contracting a disease from the door handle increases.

In view of the above, the Inventors have recognized the advantages of a door handle capable of purging itself of microorganisms, particularly disease causing microorganisms. In some embodiments, a door handle includes one or more light sources. The one or more light sources may emit light at a wavelength suitable to inhibit the growth or action of microorganisms (i.e. act as a germicidal to disinfect surfaces). In some embodiments, the one or more light sources emit light in the ultraviolet C (“UV-C”) light range, which may serve to kill, render harmless, or make less harmful some bacteria, viruses, and other pathogens, destroying or inhibiting their ability to multiply and cause disease.

However, large doses of light in the UV-C light range may cause undesired side effects. Thus, it may be desirable to minimize exposure to UV-C light.

In view of the above, the Inventors have balanced the need to neutralize microorganisms actively, while limiting exposure to UV light. For example, the Inventors have recognized the advantages of a door handle capable of selectively emitting UV-C light to sanitize the door handle. Particularly, it may be beneficial for a door handle capable of emitting germicidal light with a programmable controller or other suitable device to selectively turn the UV-C light on and off, for example when the light might otherwise be in the presence of a human.

According to one aspect, a germicidal door handle includes a body with one or more light sources mounted in the body. The one or more light sources may mount to the body via a printed circuit board or other suitable medium. In some embodiments, the printed circuit board is flexible so as to confirm to the shape of the body. For example, the body may be a flat panel, a curved surface or other suitable shape, and the printed circuit board may flex to complement the shape of the body. The printed circuit board may also act as a conduit to power the one or more light sources.

The one or more light sources may be configured to emit light at a wavelength effective to act as a germicidal. For example, the one or more light sources may emit UV-C light to act as a germicidal. In some embodiments, the one or more light sources emit light at a wavelength between 100 nm and 400 nm. Of course, the one or more light sources may emit light at any wavelength suitable for a germicidal application, including wavelengths of greater than 400 nm and less than 100 nm.

A given light source of the one or more light sources may be equipped with a light well. The light wells may serve to direct the light emitted by a given light source along a predetermined path. A fully assembled germicidal door handle may include a plurality of light wells, where each light well corresponds to one of the one or more light sources. Accordingly, the plurality of light wells may direct light from the one or more light sources along a plurality of predetermined paths. In some embodiments, a light well directs light from the given light source in a generally conical path. In some embodiments, the light well directs light from a given light source in a generally conical shape extending between 15 and 165 degrees relative to the plane of the light source. Of course, the light well may be configured direct light from the given light source in other paths such as a generally spherical path, a generically cylindrical path, or any other suitable path. Further, the predetermined path may encompass any suitable angle range relative to a plane of the given light source, including ranges between less than 15 degrees and greater than 165 degrees, depending on the application. The plurality of predetermined paths may be configured to overlap to ensure full coverage of the target surface.

In some embodiments, the one or more light sources are internal of the door handle. For example, the one or more light sources may reside within the door handle. Thus, light may shine out of the door handle via the internal functional components. Of course the one or more light sources may alternatively be positioned outside of the door handle and be configured to shine light onto the door handle externally, as other suitable configurations are also contemplated.

In some embodiments, a germicidal door handle includes a grip sleeve. The grip sleeve may be transparent to allow light from the one or more light sources to travel through the grip sleeve. Thus, the one or more light sources may serve to disinfect the grip sleeve, as described above. The grip sleeve may cover a surface of the door handle that a user may touch. For example, the grip sleeve may cover the full length of a door handle lever, the full length of a door handle push bar, the full length of a door knob, or the full length of any other suitable door handle device. Thus, the one or more light sources may be configured to disinfect every surface that a user may touch. Further, the plurality of light wells may be configured such that the plurality of light wells directs light from the one or more light sources along a plurality of paths covering the full length of the grip sleeve in the aggregate.

Generally speaking, UV-C light is not visible to the naked eye. Thus, it may be desirable for a germicidal door handle to be equipped with at least one visible light source. The visible light source may serve as an indicator. For example, the visible light source may be on when the one or more UV-C light sources are active. Further, the visible light source may be off when the one or more UV-C light sources are off. In some embodiments, the visible light source is capable of emitting visible light in one or more different colors (e.g., the RGB light range). Accordingly, the visible light source may indicate that the one or more UV-C light sources are on by emitting a red light, denoting that it may not be safe for an individual to touch the door handle. The visible light source may also emit a green light to denote that the one or more UV-C light sources are off, meaning that it is safe for an individual to touch the door handle. Of course, the visible light source may serve any other appropriate application, such as indicating a status of the door (e.g., locked or unlocked). In some embodiments, the visible light source illuminates the grip sleeve.

In some embodiments, the one or more light sources are powered by an external power supply. The external power supply may be electrically connected to an electrode in the door handle. The electrode may then provide power to the printed circuit board, which may then provide power to each of the one or more light sources.

According to another aspect, a door exit device includes a door handle, a locking mechanism, and a controller. The door handle may be configured as described above, with a body having one or more germicidal light sources mounted in the body. Other suitable surfaces that are touched by users may also include one or more germicidal light sources. Such surfaces may include a push plate, a door knob, a door/drawer pull, a door handle, a key hole, a door locking mechanism, or any other suitable surface.

The controller may include features to selectively activate the one or more light sources. For example, the controller may initially activate the one or more light sources. The controller may then deactivate the one or more light sources upon an occurrence of a first predetermined condition. The controller may then reactivate the one or more light sources upon an occurrence of a second predetermined condition. One objective of including the controller may be to prevent a human user from receiving excess exposure to potentially harmful germicidal light. Thus, the predetermined conditions may be triggered by the presence or absence of a human. For example, the controller may include a sensor (e.g., an infrared motion sensor, a camera, an ultrasonic detector, or other suitable sensor) configured to detect the presence of a human in proximity to the door exit device. When the sensor detects a human in proximity to the door exit device, the sensor may provide a first indication of the presence of the human to the controller. In turn, the first indication may trigger the first predetermined condition, causing the controller to deactivate the one or more light sources. The sensor may also be capable of providing the controller with a second indication of the absence of a human in proximity to the controller. The second indication may, in turn, trigger the second predetermined condition, causing the controller to reactivate the one or more light sources. The controller may repeat these steps as the sensor continues to provide the first and second indications to the controller as appropriate.

Of course, the first and second predetermined conditions need not be related to the presence and absence of a human in proximity to the door exit device. For example, the controller may include a timer configured to provide the controller with an indication of the passage of a predetermined time interval. In some embodiments, the passage of a predetermined time interval triggers both the first and second predetermined conditions. Thus, the controller may cycle the one or more light sources on and off, for example, to clean the door handle on a regular basis or during hours when it is known that humans will not be in proximity of the door handle (e.g., after hours in an office building). Of course, the first and second predetermined conditions may be any combination of suitable predetermined conditions. For example, in some embodiments, the first predetermined condition may be triggered by the presence of a human in proximity to the door handle, and the second predetermined condition may be triggered by the passage of a predetermined time interval.

As will be appreciated by one of skill in the art, the first and second predetermined conditions may take on any suitable configuration for a given application. For example, the first predetermined condition may be triggered by a human providing credentials to unlock the door handle. Alternatively, the first predetermined condition may be triggered by a key entering a lock of the door handle and the second predetermined condition may be triggered by the key leaving the door handle. Of course, other predetermined conditions are also contemplated.

According to another aspect, an upgrade kit for an existing door handle includes a body, one or more germicidal light sources configured to be mounted in the body, a transparent grip sleeve configured to surround the one or more light sources, and a controller as described above. The controller of the upgrade kit may be configured to be mounted on the body, for example, via a printed circuit board. The upgrade kit may also include a portable power supply. The portable power supply may be configured to couple to the body via the printed circuit board.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

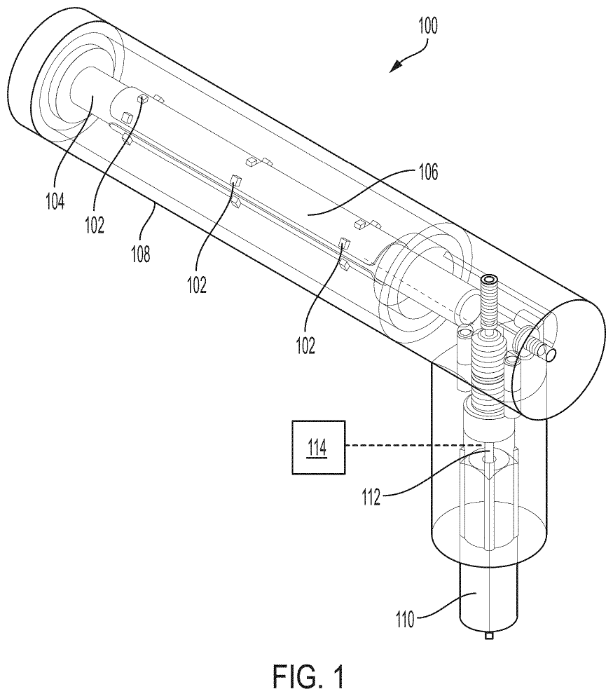

FIG. 1 is a perspective view of a germicidal door handle lever 100 according to one illustrative embodiment. Door handle lever 100 includes a plurality of light sources 102 mounted on a body 104. The plurality of light sources 102 is electronically connected to a printed circuit board 106. In one embodiment, the printed circuit board 106 is flexible and accordingly may conform its shape to the shape of body 104. Alternatively, printed circuit board 106 may be formed as a cylinder. A grip sleeve 108 surrounds the plurality of light sources 102. Grip sleeve 108 is made of a transparent material, such as clear glass, polycarbonate, acrylic, cyclic olefin copolymer, or any other suitable transparent material. Thus, light emitted by plurality of light sources 102 may shine through grip sleeve 108. In some embodiments, plurality of light sources 102 emits light at a wavelength sufficient to act as a germicidal. Thus, as indicated above, the light from plurality of light sources 102 neutralizes microorganisms on at least a portion of grip sleeve 108.

The plurality of light sources 102 may be configured in any suitable manner. For example plurality of light sources 102 may be a set of light emitting diodes, a set of excimer lamps, a set of halogen bulbs, a set of iridescent bulbs, a set of fluorescent bulbs, or any other suitable type of light source.

The plurality of light sources 102 may be powered by an external power supply 114. External power supply 114 may be in electronic communication with an electrode 112. Electrode 112 may, in turn, be in electronic communication with printed circuit board 106. The plurality of light sources 102 may be mounted on printed circuit board 106 such that plurality of light sources 102 are in electronic communication with printed circuit board 106. Thus, electricity may flow from power supply 114 to electrode 112 to printed circuit board 106 to plurality of light sources 102, providing electrical power plurality of light sources 102. Electrode 112 may be made of copper, aluminum, or any other suitable electrically conductive material.

Door handle lever 100 may interface with a locking mechanism of a door via a spindle 110. For example, as door handle lever 100 is turned, spindle 110 may turn, allowing the door to be opened. Of course, the door handle may be a simple door pull or push surface and need not be part of a door lock.

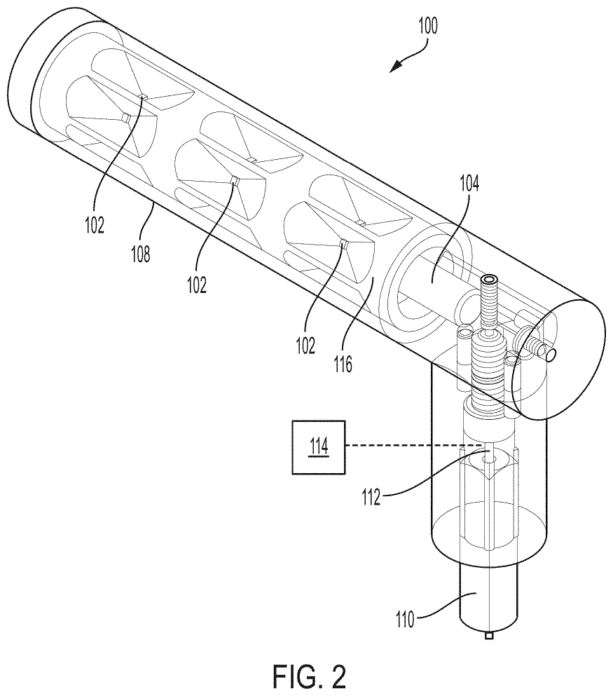

FIG. 2 is a perspective view of door handle lever 100 according to one illustrative embodiment. In this embodiment, door handle lever 100 includes a plurality of light wells 116 (in addition to plurality of light sources 102, body 104, printed circuit board 106, which are disposed under plurality of light wells 116 in FIG. 2, and grip sleeve 108). Plurality of light wells 116 may be configured to direct light from plurality of light sources 102 along a predetermined path. In some embodiments, each individual light source of plurality of light sources 102 includes a corresponding light well of plurality of light wells 116. Thus, plurality of light wells 116 may be configured to direct the light emitted by plurality of light sources 102 in along a plurality of paths covering the entirety of grip sleeve 108. Accordingly, the light emitted by plurality of light sources 102 may be directed by plurality of light wells 116 to travel along a predetermined path such that the light emitted by plurality of lights 102 disinfects the entirety of grip sleeve 108. Further, grip sleeve 108 may encompass every surface that a user is likely to touch during operation of the door.

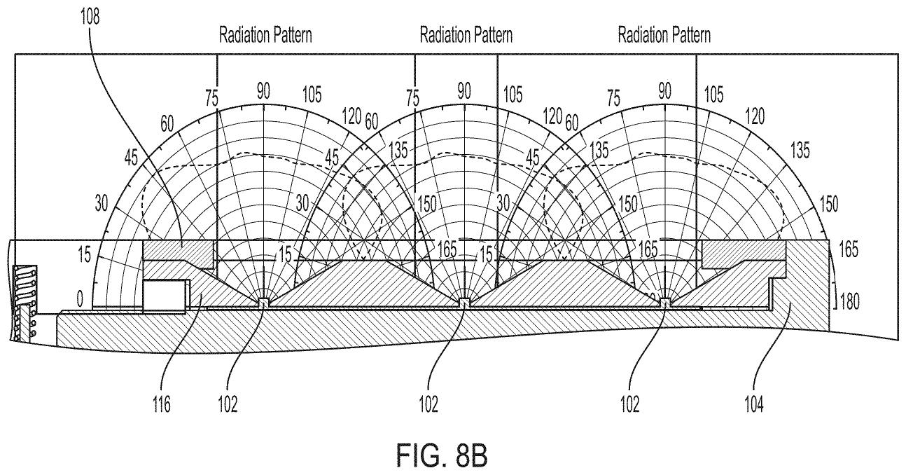

Plurality of light wells 116 may be configured to direct light emitted by plurality of light sources 102 along any suitable path. For example, FIG. 8A shows one light well of plurality of light wells 116 directing light from one light source of plurality of light sources 102 along a generally conical path, according to one embodiment. For example, a light well may direct light emitted by a light source along a generally conical path between 15 and 165 degrees relative to a plane of the light source. Of course, the light well may direct light along any suitable path, including paths between less than 15 degrees relative to the plane of the light source and greater than 165 degrees relative to the plane of the light source.

In some embodiments, plurality of light wells 116 may direct light from plurality of light sources 102 in an overlapping pattern, as shown in FIG. 8B. In some embodiments, the individual light wells of plurality of light wells each direct light along similarly shaped paths, though this need not be the case. For example, one individual light well of plurality of light wells 116 may direct light in a generally conical shape while another light well of plurality of light wells 116 may direct light in a generally cylindrical path. In some embodiments, plurality of light wells 116 is configured to direct light in a plurality of patterns that covers the full surface area of grip sleeve 108 in the aggregate.

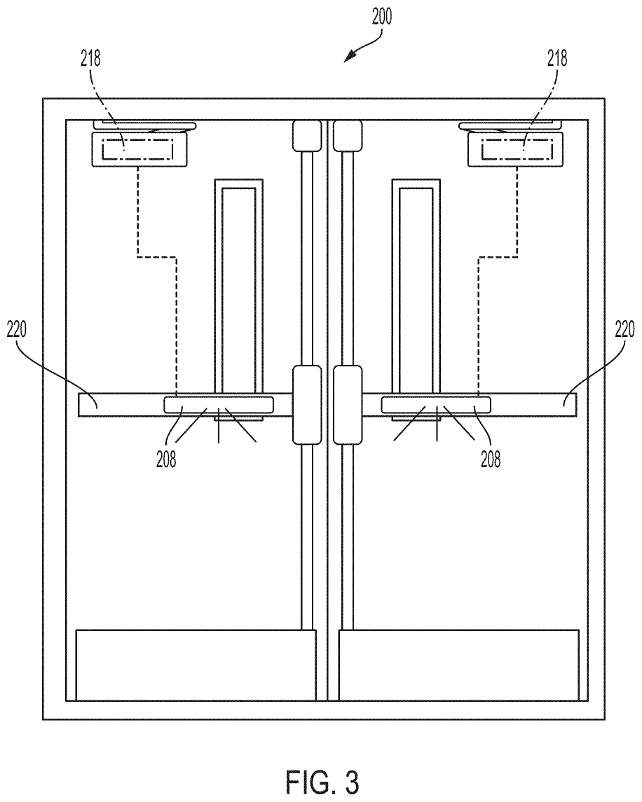

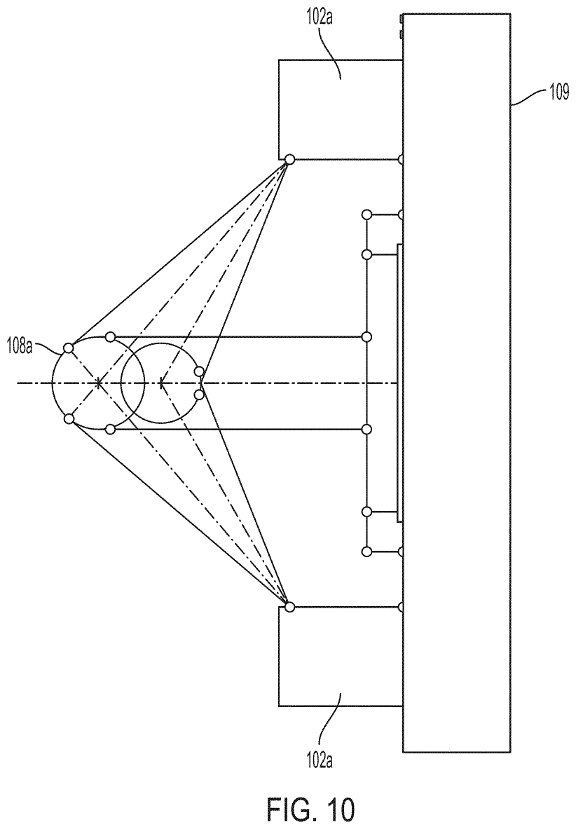

Of course, the light source(s) may be configured to shine light onto the surface to be touched by a human. For example, FIG. 10 shows an embodiment of an access control system including one or more light sources 102a arranged to shine light onto a handle surface, such as a grip sleeve 108a, mounted to a door 109. The plurality of light sources should be of sufficient quantity to cover the surfaces to be touched by the human user. In the depicted embodiment, the light sources 102a surround and shine light onto grip sleeve 108a. The light source(s) shine light in a generally radial direction. Alternatively, light source(s) 102a may be configured to shine light onto grip sleeve 108a in a generally axial direction. Of course, any suitable configuration of external light sources may be used. In embodiments where light source(s) 102a is located externally to touchable surface, the touchable surface, as in grip sleeve 108a, may be transparent, translucent, or opaque. Thus, in addition to clear glass, polycarbonate, acrylic, cyclic olefin copolymer, the touchable surface may be made of plastic, metal, wood, frosted glass, or other suitable material. P FIG. 3 shows a front view of a door exit device 200 according to one illustrative embodiment. Door exit device 200 may include an actuator such as push bar 220. Push bar 220 may include an actuation surface 208 through which one or more light sources may emit light having a germicidal wavelength, as described above. Thus, door exit device may include features to neutralize microorganisms on actuation surface 208. The functional components of actuation surface 208 may be disposed within actuation surface 208.

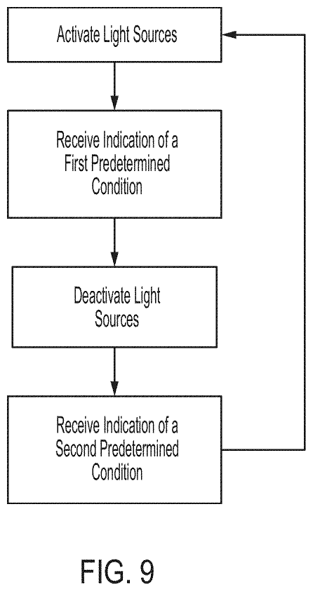

The functional components (e.g., the one or more light sources) of any of the embodiments described herein may be in electronic communication with a controller 218. Accordingly, controller 218 may include features that allow controller 218 to control the one or more light sources disposed within actuation surface 208, grip sleeve 108, or any other suitable medium. For example (as shown in FIG. 9), controller 218 may initially turn on the one or more light sources. Subsequently, upon receiving a first indication, controller 218 may turn off the one or more light sources until controller 218 receives a second indication. Upon receiving the second indication, controller 218 may then turn the one or more light sources back on.

Controller 218 may be configured in any suitable manner. For example controller 218 may be configured as a continuous controller, a discontinuous controller, a two-position controller, a multi-position controller, a proportional controller, an integral controller, a derivative controller, or any other suitable controller configuration, depending on the application.

Controller 218 may be located outside of actuation surface 208, grip sleeve 108, or other suitable medium. In some embodiments, controller 218 may be part of door exit device 200, as shown in FIG. 3, though controller 218 may also be part of a door handle lever 100 as shown in FIG. 1. Though controller 218 may be located outside of actuation surface 208 (or grip sleeve 108), controller 218 may electronically communicate with the functional components of the germicidal lighting apparatus within actuation surface 208 (or grip sleeve 108) via either a wired or wireless connection. Controller 218 and the functional components of the germicidal lighting apparatus within actuation surface 208 (or grip sleeve 108) may be powered by a power supply located on door exit device 200 (or door handle 100).

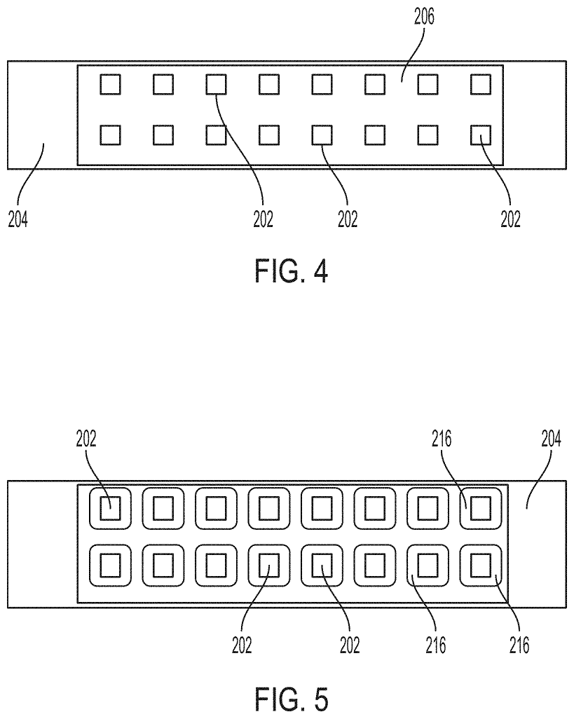

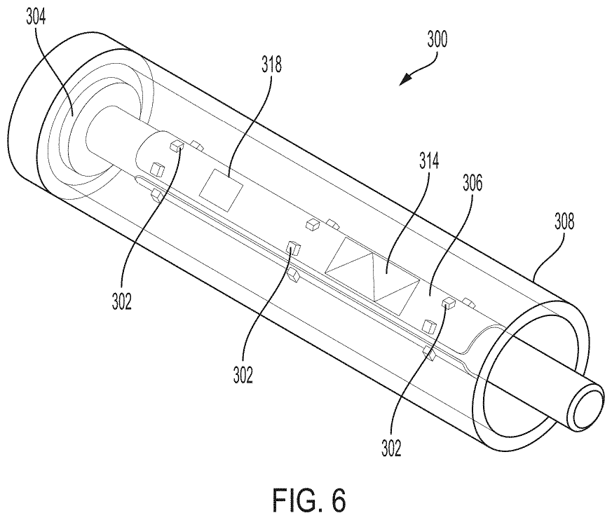

FIG. 4 shows the functional components of a germicidal lighting apparatus to be used within actuation surface 208 of door exit device 200, according to one illustrative embodiment. As with the functional components of door handle 100, a plurality of light sources 202 may be disposed on a flexible printed circuit board shaped 206 to surround a body 204. As shown in FIG. 5, the functional components of a germicidal lighting apparatus to be used within actuation surface 208 of door exit device 200 may further include a plurality of light wells 216 configured to function similarly to the plurality of light wells 116 of door handle 100.

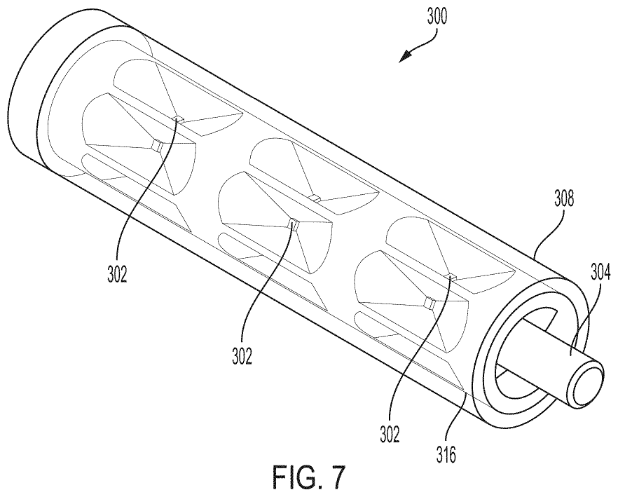

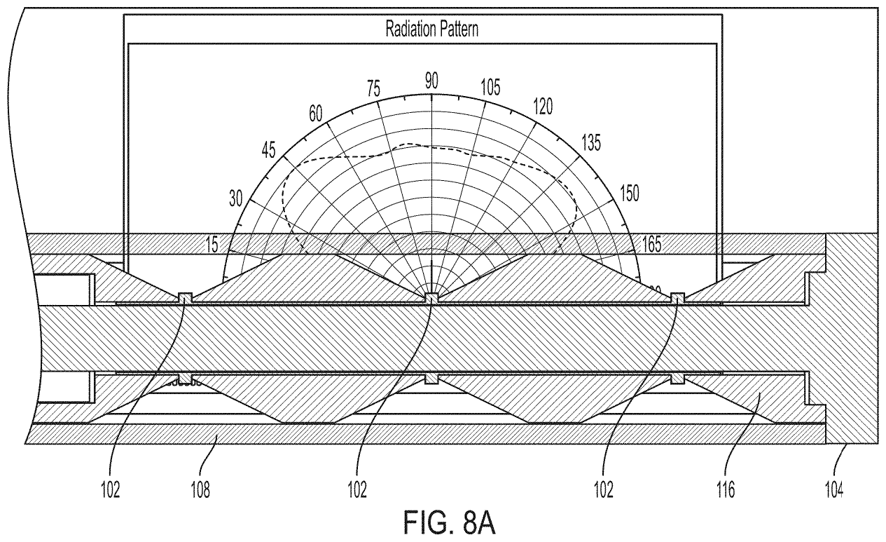

FIG. 6 shows a germicidal door handle upgrade kit 300 configured to replace a preinstalled door handle, according to one illustrative embodiment. Particularly, upgrade kit 300 may be configured to interface with an existing locking mechanism of a door. Upgrade kit 300 may include a plurality of light sources 302 disposed on a body 304 via a printed circuit board 306. Upgrade kit 300 may also include a transparent grip sleeve 308 configured to surround plurality of light sources 302, printed circuit board 306 and body 304. As with the previously discussed embodiments, plurality of light sources 302 may be configured to emit light at a wavelength suitable to act as a germicidal. Thus, plurality of light sources 302, and body 304 may be configured similarly to plurality of light sources 102 and body 104 of door handle 100. Printed circuit board 306 may include a controller 318 and a portable power source 314. In some embodiments, controller 318 and portable power source 314 may be mounted on printed circuit board 306 such that power source 314 and controller 318 do not obstruct the light emitted by plurality of light sources 302. Power source 314 may provide power to plurality of light sources 302 via printed circuit board 306. In some embodiments, controller 318 is configured to perform the same steps as controller 218 of door exit device 200.

Portable power source 314 may be configured in any suitable manner. For example, portable power supply 314 may be a battery. Accordingly, portable power supply may be a Lithium-ion battery, a Nickel-Cadmium battery, a Lead-acid battery, a molten salt battery, a galvanic cell, a dry cell, a Zinc-air battery, or any other suitable battery. Power source 314 may also be configured with any suitable capacity. For example, power source 314 may be configured with a capacity of 250 amp-hours, 350 amp-hours, or 500 amp-hours. Of course, power source 314 may be configured with a range of suitable capacities including capacities of less than 250 amp-hours, between 250 and 350 amp-hours, between 350 and 500 amp-hours, and greater than 500 amp-hours, depending on the application.

Example: UV-C Light Source Parameter Optimization for Purging COVID-19

Through experimentation and modeling, the Inventors have determined parameters for a door handle lock including a germicidal lighting apparatus optimized for purging COVID-19 from the door handle, while still allowing the door handle to be safe for human use. For example, the one or more light sources may be configured to emit UV-C light with a radiant power output of 2.6 mW at 100% power (approximately 20 mA@5V). Without being bound by theory, radiant energy exposure may be represented as (power output×duration)/distance\hat{ }2. The radiant energy exposure may represent the accumulated exposure at a point some known distance from the one or more light sources. Thus, applying the formulation above, if a given light source is active for 30 seconds and the output is measured lm away from the light source, the output radiant energy exposure is 0.078 J/m\hat{ }2.

There are a number of known guidelines that dictate the radiant energy exposure that is considered safe for humans. One such guideline lists a safe exposure as 30 J/m\hat{ }2 over the course of an 8 hour workday.

Applying the formula described above, the Inventors calculated the safe distance for a human relative to the light source. Each time a human approaches a germicidal door handle, the human may be exposed to UV-C radiation for 10-20 seconds per use. Accordingly, a door that has 100 uses per day will have 10 times the exposure as a door that has 10 uses per day.

Without being bound by theory, the total accumulated exposure for a user may be represented as radiant energy exposure for 1 cycle×N, where N is the number of uses per day. In view of the above and without being bound by theory, safe distance for a human user may be represented as follows: sqrt((Power×Duration×N)/(max safe exposure)). If during a single use, the human is exposed to UV-C radiation for 20 seconds, and the human opens the door 100 times in one day, applying the formula above, the safe distance would be 0.416 meters (1.36 ft). Accordingly, a human may safely stand 1.36 feet away from the door while the UV-C lights are active.

Data exist that specify the radiant energy exposure required to kill a certain percentage of COVID-19 on a surface. One such report shows a 90% reduction in COVID-19 at 6.11 J/m\hat{ }2.

Without being bound by theory, the Inventors calculated how long COVID-19 needs to be exposed to UV-C light to reduce the presence of COVID-19 by 90% using the following formula: Duration=(energy exposure×distance\hat{ }2)/(Power output).

In some embodiments, the farthest point of the door handle from the light source is ⅝ inches (0.0159 m). Applying the formula above, the virus needs to be exposed to UV-C radiation for 0.594 seconds for a 90% reduction in COVID-19 at a distance of ⅝ inches.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computing device or distributed among multiple computing devices. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Also, the controller may have one or more input and output devices. These devices can be used, among other things, to present a user interface. Examples of output devices that can be used to provide a user interface include display screens for visual presentation of output and speakers or other sound generating devices for audible presentation of output. Examples of input devices that can be used for a user interface include keyboards, individual buttons, and pointing devices, such as mice, touch pads, and digitizing tablets. As another example, a computing device may receive input information through speech recognition or in other audible format.

Such controllers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the embodiments described herein may be embodied as a computer readable storage medium (or multiple computer readable media) (e.g., a computer memory, one or more floppy discs, compact discs (CD), optical discs, digital video disks (DVD), magnetic tapes, flash memories, RAM, ROM, EEPROM, circuit configurations in Field Programmable Gate Arrays or other semiconductor devices, or other tangible computer storage medium) encoded with one or more programs that, when executed on one or more computers or other processors, perform methods that implement the various embodiments discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computing devices or other processors to implement various aspects of the present disclosure as discussed above. As used herein, the term “computer-readable storage medium” encompasses only a non-transitory computer-readable medium that can be considered to be a manufacture (i.e., article of manufacture) or a machine. Alternatively or additionally, the disclosure may be embodied as a computer readable medium other than a computer-readable storage medium, such as a propagating signal.

The terms “program” or “software” are used herein in a generic sense to refer to any type of computer code or set of computer-executable instructions that can be employed to program a computing device or other processor to implement various aspects of the present disclosure as discussed above. Additionally, it should be appreciated that according to one aspect of this embodiment, one or more computer programs that when executed perform methods of the present disclosure need not reside on a single computing device or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A door handle lever comprising:
a body;
one or more light sources mounted in the body;
a transparent grip sleeve surrounding the one or more light sources;
wherein the one or more light sources are configured to emit light at a wavelength effective to act as a germicidal.

2. The door handle lever of claim 1, further including a printed circuit board mounted in the body, wherein the one or more light sources are mounted to the printed circuit board.

3. The door handle lever of claim 1, further comprising a light well surrounding a corresponding light source.

4. The door handle lever of claim 3, wherein the light well is configured to direct light from the corresponding light source along a predetermined path.

5. The door handle lever of claim 4, wherein the one or more light sources comprises a plurality of light sources and wherein the light well comprises a plurality of light wells, each light well corresponding to one light source, wherein the plurality of light wells direct light from the plurality of light sources along a plurality of predetermined paths, wherein the plurality of predetermined light paths is configured such that the light emitted by the plurality of light sources covers an entire surface area of the grip sleeve.

6-7. (canceled)

8. The door handle lever of claim 5, wherein the grip sleeve is configured to cover a surface of the door handle lever that a user may touch when actuating the door handle lever.

9. The door handle lever of claim 1, wherein the one or more light sources are configured to emit light at a wavelength between approximatelyl00 nm and approximately 400 nm.

10. (canceled)

11. The door handle lever of claim 1, wherein the one or more light sources includes a light source configured to emit visible light.

12. A door exit device comprising:
a door handle;
a locking mechanism;
a body;
one or more light sources mounted in the body;
a transparent grip sleeve surrounding the one or more light sources;
a controller disposed on the locking mechanism, wherein the controller performs the steps of:
activating the one or more light sources;
deactivating the one or more light sources upon an occurrence of a first predetermined condition;
reactivating the one or more light sources upon an occurrence of a second predetermined condition; and
wherein the one or more light sources are configured to emit light at a wavelength effective to act as a germicidal when active.

13. The door exit device of claim 12, further including a printed circuit board mounted in the body, wherein the one or more light sources are mounted to the printed circuit board.

14. The door exit device of claim 12, further comprising a light well surrounding a corresponding light source.

15. The door exit device of claim 14, wherein the light well is configured to direct light from the corresponding light source along a predetermined path.

16. The door exit device of claim 15, wherein the one or more light sources comprises a plurality of light sources and wherein the light well comprises a plurality of light wells, each light well corresponding to one light source, wherein the plurality of light wells direct light from the plurality of light sources along a plurality of predetermined paths, wherein the plurality of predetermined light paths is configured such that the light emitted by the plurality of light sources covers an entire surface area of the grip sleeve.

17.-18. (canceled)

19. The door exit device of claim 16, wherein the grip sleeve is configured to cover a surface of the door exit device that a user may touch when actuating the door handle.

20. The door exit device of claim 12, wherein the one or more light sources are configured to emit light at a wavelength between approximately 100 nm and approximately 400 nm when active.

21. (canceled)

22. The door exit device of claim 12, wherein the controller includes a motion sensor configured to provide a first indication of the presence of a human in proximity to the door exit device.

23. The door exit device of claim 22, wherein the motion sensor is configured to provide a second indication of the absence of a human in proximity to the door exit device.

24. The door exit device of claim 23, wherein the first indication of the presence of a human in proximity to the door exit device triggers the first predetermined condition.

25. The door exit device of claim 24, wherein the second indication of the absence of a human in proximity to the door exit device triggers the second predetermined condition.

26.-27. (canceled)

28. The door handle lever of claim 12, wherein the one or more light sources includes a light source configured to emit visible light.

29.-45. (canceled)

46. An access control system for an entryway, the system comprising:
a surface configured to be touched by a user and configured to assist in access through the entryway;
one or more light sources mounted adjacent to the surface;
wherein the one or more light sources are configured to emit light onto the surface at a wavelength effective to act as a germicidal.
